(19) United States
(12) Patent Application Publication
(10) Pub. No.: US 2026/0124472 A1
(43) Pub. Date: May 7, 2026

(54) TREATMENT DEVICE

(71) Applicant: NEUMIC THERAPEUTICS LIMITED, Dunedin (NZ)

(72) Inventors: John Noble James REYNOLDS, Dunedin (NZ); Paul David HARRIS, Lower Hutt (NZ); Stephen John SOWERBY, Dunedin (NZ)

(21) Appl. No.: 18/704,312

(22) PCT Filed: Oct. 27, 2022
(86) PCT No.: PCT/NZ2022/050131

§ 371 Date: Apr. 24, 2024

Related U.S. Application Data
(60) Provisional application No. 63/273,041, filed on Oct. 28, 2021.

(51) Int. Cl. A61N 7/00

(52) U.S. Cl. CPC A61N 7/00; A61N 2007/0078

(57) ABSTRACT

This invention relates to a treatment device, the use of a treatment device, and the manufacture of a treatment device, particularly in the treatment of brain disorders.

### FIELD

This invention relates to a treatment device, the use of a treatment device, and the manufacture of a treatment device, particularly for the treatment of brain disorders.

### BACKGROUND

Treatment of the mammalian brain for disorders, diseases, injuries, and research is complicated by the complexity of the brain, the protection of the bones of the skull, and the selective permeability of the sieve-like tissue layer called the blood brain barrier (BBB) which encircles each blood vessel and separates the contents within the blood vessels from the neurons of the brain.

Drugs introduced into the blood stream to treat the brain are systemic to the body's whole circulatory system and, when entering the blood vessels of the brain, may pass through the BBB and distribute throughout the whole brain, with little control of the location or timing of their activity. The systemic administration of drugs intended to target specific regions of the brain may cause detrimental side-effects on non-target areas of the brain and of the body.

Technologies have been developed to deliver types of radiation to volumes inside the skull to ameliorate at least some of these problems. To date, these devices are typically applied to the head by large instruments in which participants are held immobile. The individual to receive the radiation is mechanically conveyed into the region of the instrument where the radiation is emitted. This approach has the disadvantage that such instruments are large, expensive, located in clinics, and are under high demand, meaning long waiting times between consecutive applications. Treatments are imposing for the participants and may not be practically able to apply continuous or semi continuous or on-demand signals to specific volumes of the brain over extended periods of time.

Furthermore, instruments that deliver radiation to specific volumes of the brain often require a stereotactic frame to be surgically installed on the head of the participant to immobilize the head during the delivery of radiation. This can be uncomfortable and undesirable for the participant, introduces surgical risks, and adds further costs.

There is a need for a treatment device capable of delivering ultrasound pressure and/or photonic illumination and/or magnetic fields to specific volumes inside the skull that is without the disadvantages of large instruments, implants, or indwellings, which can provide continuous use or semi-continuous or on-demand use, is able to be conveniently reconfigured to alternative volumes inside the skull, is widely available, is conveniently maintained, and is comparatively inexpensive.

### SUMMARY

According to one example there is provided a treatment device that can be configured to be worn on a head and can comprise a frame configured to be worn on a head, and a plurality of emitters supported by the frame, the plurality of emitters configured to deliver a first modality of radiation to a volume within the head; wherein the plurality of emitters is configured to activate a medicament within the volume irradiated by the emitters.

According to another example there is provided a treatment device that can be configured to be worn on a head and can comprise a frame configured to be worn on a head; and a plurality of emitters supported by the frame, each emitter being configured to deliver at least one modality of radiation to a volume within the head; wherein the treatment device is configured so that an orientation and/or position of at least one emitter with respect to the frame can be adjusted, and wherein the plurality of emitters is configured to deliver radiation to a common volume within the head.

According to another example there is provided a method of manufacturing a customised head-wearable treatment device that can comprise imaging a head to determine a three-dimensional topology of the head; identifying at least one specified volume within the head to be irradiated by a plurality of emitters; determining a desired orientation of at least one emitter with respect to the specified volume; and manufacturing a customised frame configured to be worn on the head; wherein the customised frame is configured to support the at least one emitter such that when the customised frame is worn on the head, the at least one emitter is positioned with the desired orientation with respect to the specified volume within the head.

According to another example of the invention there is provided a method to treat a brain disorder in a subject in need thereof comprising applying a treatment device as described herein to the head of said subject.

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning-i.e., they will be taken to mean an inclusion of the listed components which the use directly references, and possibly also of other non-specified components or elements.

Reference to any document in this specification does not constitute an admission that it is prior art, validly combinable with other documents or that it forms part of the common general knowledge.

### BRIEF DESCRIPTION OF THE DRAWINGS

The accompanying drawings which are incorporated in and constitute part of the specification, illustrate examples of the invention and, together with the general description of the invention given above, and the detailed description of examples given below, serve to explain the principles of the invention, in which:

FIG. 1A depicts an example of a treatment device;

FIG. 1B depicts an example of an emitter;

FIG. 2 depicts a further example of a treatment device;

FIG. 3 depicts a further example of a treatment device;

FIG. 4 depicts a further example of a treatment device;

FIG. 5 depicts a further example of a treatment device;

FIG. 6 depicts a method of manufacturing a treatment device;

FIG. 7 depicts an example of a treatment device;

FIG. 8 depicts a recording made by a hydrophone;

FIG. 9 depicts a contour map showing a measured sound pressure level;

FIG. 10 depicts a contour map showing a measured sound pressure level;

FIG. 11 depicts a desired orientation of a number of emitters with respect to a phantom head;

FIG. 12 depicts a treatment device on a phantom head; and

FIG. 13 depicts a treatment device on a phantom head.

### DETAILED DESCRIPTION

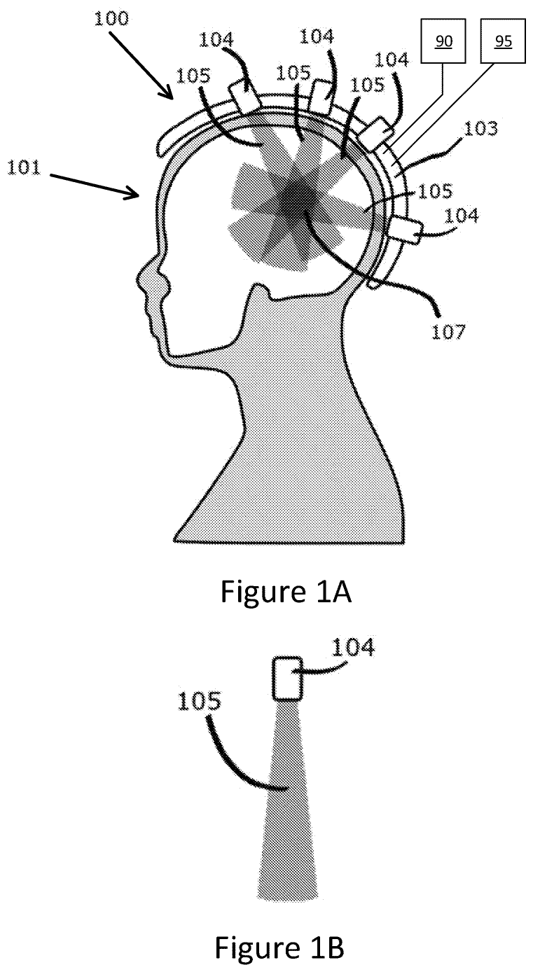

FIG. 1A depicts an example of a treatment device 100. The treatment device 100 includes a frame 103 that is configured to be worn on a head of a user 101. The frame 103 supports a plurality of emitters 104 that are configured to deliver at least one modality of radiation, schematically depicted as beams 105, to a volume 107 within the head of the user 101. The plurality of emitters 104 is configured to activate a medicament within the volume 107 that is irradiated by the plurality of emitters 104. The medicament may be thermally activated, mechanically activated, and/or magnetically activated. For example, the medicament may be a thermally activated liposome.

The treatment device 100 further includes (or is in operative communication with) control circuity 90 that is used to control the operation of the emitters 104 and power circuity 95 that provides power to the treatment device 100.

‘Radiation’ as used herein refers to the emission, transmission, and/or propagation of energy in the form of waves or particles, including acoustic radiation (such as ultrasonic radiation) and electromagnetic radiation (such as infrared radiation.) The modality of radiation emitted by the plurality of emitters 104 can depend on the application of the treatment device 100 (e.g. the type of medicament that is to be activated within volume 107). For example, the plurality of emitters 104 can be configured to emit ultrasonic/focused ultrasonic, electromagnetic radiation (such as infrared or near-infrared radiation), or magnetic fields. Any or all of the emitters 104 may be adjustable as described in more detail below.

In some examples, the emitters 104 are selected from the group comprising: acoustic emitters; ultrasound emitters; electromagnetic emitters; light emitters; radio emitters; magnetic emitters; and/or magnetic field emitters. A single emitter 104 and its emission 105 are displayed in FIG. 1B.

In some examples, the frame 103 can be selected from a group comprising permanent frames; adjustable frames; bespoke frames; flexible frames; rigid frames; metallic frames; polymer frames; composite frames; frames made by additive manufacturing; and/or frames made by 3D-printing.

The treatment device 100 depicted in FIG. 1A includes emitters 104 that are positioned around frame 103 such that the irradiated volume 107 receives radiation from each emitter 104. This arrangement reduces the amount of radiation received by off-axis volumes within the head of the wearer 101. This can improve the precision of treatment and can reduce detrimental outcomes that can arise when non-target volumes are irradiated, such as thermal lesioning. The emitters 104 are usually positioned about the frame 103 so that the beams 105 are approximately orthogonally to the target volume 107.

It is noted that the direction and shape of the radiation depicted in FIG. 1A by beams 105 is only schematic, and the actual shape of the beam of radiation emitted by each emitter 104 may have a more complex path from emitter 104 to target volume 107. For example, ultrasonic radiation may reflect and/or refract between or through different tissues within the head of the user 101 due to differences in impedance. The front of the beam 105 may also broaden out so that its direction is somewhat less defined.

In some examples, the emitters 104 are positioned about the frame 103 so that their emissions are: focused; defocused; orthogonal; colinear; overlapping; intersecting; and/or superimposed.

In some examples, the plurality of emitters 104 can be configured in an array. In some examples, the array of emitters is selected from the group comprising: sparse arrays; dense arrays; arrays of less than 3 emitters; arrays of less than 10 emitters; arrays of less than 20 emitters; arrays of less than 50 emitters; arrays of less than 100 emitters; and/or arrays of less than 300 emitters. Sparse arrays of emitters can be advantageous in that specified volumes inside the skull are not dominated by a single emitter, which would make targeting very dependent on the placement of the treatment device. There can be a trade-off between the number of emitters 104 and the illumination volume 107, acceptability of aberrations in the illumination field due to the incomplete coverage of the head, the physical weight of the treatment device, and manufacturing costs of the treatment device.

In some applications of the treatment device 100, the volume 107 that is irradiated by the emitters 104 can include one or more of: tissue; scalp; bone; dura mater; arachnoid mater; pia mater; brain tissue; grey matter; white matter; blood vessels; vasculature; neurons; glial cells; cerebral hemisphere; cerebellar hemisphere; lobe; frontal lobe; parietal lobe; temporal lobe; occipital lobe; cortex; frontal cortex; motor cortex; sensory cortex; occipital cortex; insula cortex; temporal cortex; cerebellum; brainstem; midbrain; pons; medulla; diencephalon; thalamus; hypothalamus; striatum; caudate nucleus; putamen; globus pallidus; subthalamic nucleus; substantia nigra; amygdala; hippocampus; drugs; malignant tissue; tumor; diseased tissue; gliosis; neuromodulator drugs; cytotoxic drugs; contrast agents; ultrasound responsive materials; ultrasound labile materials; photo responsive materials; magnetic field responsive materials; phase change materials; bubbles; gas bubbles; liquid bubbles; perfluorocarbons; lipids; membranes; micelles; lipid bilayers; liposomes; solid lipid nanoparticles; cubisomes; solid particles; solid nanoparticles; hollow nanoparticles; metallic particles; non-metallic particles; magnetic particles; ferromagnetic particles; plasmonic particles; surface enhanced plasmonic particles; plasmonic nanoparticles; gold particles; gold nanoparticles; inorganic particles; amorphous particles; crystalline particles; semi-crystalline particles; particles tethered to lipids; particles encapsulated by lipids; molecules; proteins; antibodies; peptides; nucleic acids; deoxyribose nucleic acids; ribose nucleic acids; genetic constructs; genes; gene sequences; vectors; viruses.

In some examples, the volume 107 that is irradiated by the plurality of emitters 104 is less than 1 mm³; less than 5 mm³; less than 10 mm³; less than 20 mm³; less than 50 mm³; less than 100 mm³; less than 200 mm³; less than 500 mm³; less than 1000 mm³; less than 2000 mm³; less than 5000 mm³; less than 10,000 mm³; less than 50,000 mm³; less than 100,000 mm³; 500,000 mm³; less than 1,000,000 mm³; and/or less than 2,100,000 mm3.

In some examples, the shape of the volume 107 is regular; irregular; spherical; ovoid; and/or a combination or superposition of shapes.

Although only one volume 107 is depicted in FIG. 1A, this is not intended to be limiting. Some applications of the treatment device 100 will involve the irradiation of multiple volumes within the head of the wearer 101. In some examples, the volume 107 is a number of overlapping or nonoverlapping volumes selected from the group comprising; one; two; three; four; five; six; seven; less than 20 specified volumes; less than 100 specified volumes; and/or less than 300 specified volumes.

In some examples, the emitters 104 are acoustic emitters and are selected from the group comprising: ultrasound emitters; ultrasound transducers; and/or piezoelectric transducers.

In some examples, the emitters 104 emit ultrasonic radiation at a frequency selected from the group comprising: about 200 kHz, 300 kHz, 400 kHz, 500 kHz, 600 kHz, 700 kHz, 800 kHz, 900 kHz, 1 MHz, 2 MHz, 3 MHz, 4 MHz, 5 MHz, 6 MHz, 7 MHz, 8 MHz, 9 MHz, 10 MHz, 11 MHz, 12, MHz, 13 MHz, 14 MHz, 15 MHz, 16 MHz, 17 MHz, 18 MHz, 19 MHz, 20 MHz, 21 MHz, 22 MHz, 23, MHz, 24 MHz, 25 MHz, 26 MHz, 27 MHz, 28 MHz, 29 MHz, 30 MHz, 31 MHz, 32 MHz, 33 MHz, 34 MHz, 35 MHz, 36 MHz, 37 MHz, 38 MHz, 39 MHz, 40 MHz, 41 MHz, 42 MHz, 43 MHz, 44 MHz, 45 MHz, 46 MHz, 47 MHz, 48 MHz, 49 MHz or about 50 MHz. In one example, the frequency is about 1 MHz. In another example the frequency is about 300 kHz. In another example the frequency is about 600 kHz. In other examples, the ultrasound signal is provided at a frequency of between about 50 MHz and about 100 MHz, that is at a frequency of about 50 MHz, 55 MHz, 60 MHz, 65 MHz, 70 MHz, 75 MHz, 80 MHz, 85 MHz, 90 MHz, 95 MHz or about 100 MHz.

In some examples, the emitters 104 emit ultrasonic radiation at an intensity selected from the group comprising: less than 100 Watt/cm²; less than 50 Watt/cm²; less than 10 Watt/cm²; less than 1 Watt/cm²; less than 0.1 Watt/cm²; less than 0.01 Watt/cm².

Emitters 104 that deliver ultrasonic radiation or other acoustic radiation to volume 107 inside the skull may enable imaging of the brain, lesioning portions of the brain, lesioning malignant tissues in the brain, activating neurons in the brain, repairing damage to the brain, increasing the permeability of the BBB for delivery of materials to the brain, and activating acoustic-responsive drugs to specific regions of the brain. The use of ultrasound focused to specific volumes inside the skull can provide a way to apply acoustic pressure to specific volumes of brain tissue, including neurons and blood vessels, inside the skull without the need for craniotomy.

Although the treatment device 100 is depicted on the head of a live human being in FIG. 1A, this is not intended to limit the use of the treatment device 100. In some applications, the head of the wearer can be one or more of a model; phantom; living; individual; cadaveric; representative; animal; primate; human; non-human primate; dog; sheep; horse; cow; mouse; and/or rat head.

The control circuitry 90 and power circuitry 95 may be head-mounted, tethered, and/or in wireless communication with treatment device 100. The control circuitry 90 and/or power circuity 95 can include and/or utilise power supplies; batteries; cables; electronic circuits; optoelectronic circuits; integrated circuits; microcircuits; microprocessors; electronic memory; electronic components; telecommunications; global positioning system; Bluetooth; wireless; software; firmware; internet connectivity; software; firmware; code; operating system; applications; protocols; and/or internet protocols.

In one example, the treatment device may be in communication with an external control and power unit (for example, a small backpack unit) that incorporates one or more of a power supply, electronics, motion sensors, GPS telemetry, and internet connectivity.

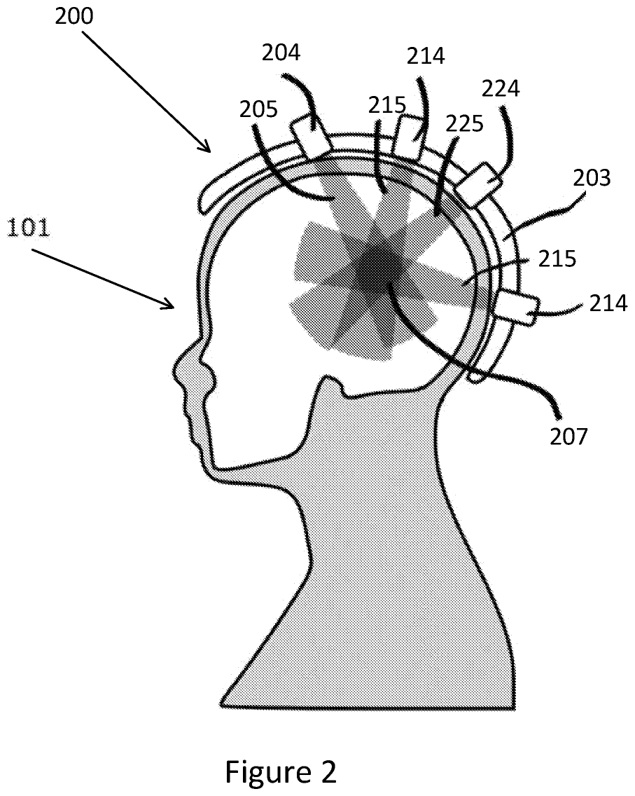

FIG. 2 depicts a treatment device 200 that includes a frame 203, a first plurality of emitters 204 that are configured to emit radiation of a first modality (depicted as beam 205), and a second plurality of emitters 214 that are configured to emit radiation of a second modality (depicted as beam 215). The treatment device 200 can alternatively include a single second emitter 214 that is configured to emit radiation of a second modality in addition to the first plurality of emitters 204. The first plurality of emitters 204 and the second plurality of emitters 214 (or second emitter 214) are configured to deliver radiation to a volume 207 within the head of the user 101.

The treatment device 200 can also include a third plurality of emitters 224 (or a third emitter 224) that is/are configured to emit radiation of a third modality (depicted as beam 225). For example, the first plurality of emitters 204 can be configured to emit ultrasonic/focused ultrasonic radiation, the second plurality of emitters 214 can be configured to emit electromagnetic radiation, and the third plurality of emitters 224 can be configured to emit magnetic fields. The respective emitters (or pluralities of emitters) are all configured to deliver radiation to a volume 207 within the head of the wearer 101 and can be configured to activate a medicament within the volume 207.

In some examples where the treatment device 200 is configured to activate a medicament (such as a liposome) within the target volume 207, the emitters 204, 214, and 224 (if applicable) of the treatment device 200 may be configured so that the emitter(s) of any one modality deliver a sub-threshold dose of radiation to the target volume 207, but the emitter(s) of both (or all) modalities deliver a threshold dose of radiation to the target volume 207.

For example, a treatment device 200 can include a first plurality of emitters 204 configured to emit focused ultrasonic radiation and a second plurality of emitters 214 configured to emit electromagnetic radiation. The net emissions of the ultrasonic emitters 204 can be below the threshold required to activate a medicament within the volume 207. Likewise, the net emissions of the electromagnetic radiation emitters 214 can also be below the threshold required to activate a medicament within the volume 207. However, the combination of the net emissions of the ultrasonic emitters 204 and the electromagnetic radiation emitters 214 can be at or above the threshold required to activate the medicament within the volume 207.

Using different modalities of radiation that are individually below the threshold required to activate the medicament within volume 207 can improve participant safety by reducing the risk of irradiating and damaging off-axis or non-target volumes within the head of the user. For example, a liposome within volume 207 may be thermally activated. The net radiation required to thermally activate the liposome could potentially cause thermal lesions if the radiation intended for volume 207 was misdirected. Splitting the net radiation into two or more sub-threshold modalities means that if the emitters of a given modality are somehow misaligned or are incorrectly configured, the off-target volumes that are irradiated by those emitters will receive a less harmful amount of radiation. This can significantly reduce the chance of thermal lesions or other damage because of the comparatively low power of any single modality of radiation.

In contrast, if the emitters of each modality are accurately aligned and properly configured, the volume 207 will receive each modality of radiation in combination, thereby providing a threshold dose of radiation to the medicament within volume 207.

Although the above example uses two modalities of radiation, the treatment device 200 can also include emitters of three modalities (for example, ultrasonic/focused ultrasonic, electromagnetic radiation, and magnetic fields) such that the emissions from a single modality are sub-threshold, but the collective modalities are at or above a threshold dose. In the case where three modalities are used, the emitters of all three modalities may be required to deliver radiation to a common volume in order to provide a threshold dose. In other examples, emitters of only two of the three modalities may be required to provide a threshold dose to volume 207.

Whilst the emitters are configured to deliver radiation to a common volume, the individual volumes that are irradiated by emitters of each modality may differ in size, shape, and location. For example, one plurality of emitters can be used to deliver diffuse sub-threshold electromagnetic radiation (e.g. infrared radiation) to a comparatively large volume within the head. A second plurality of emitters can be used to deliver sub-threshold focused ultrasonic radiation to a much smaller volume that at least partially overlaps or intersects with the comparatively large volume. The intersection of the larger volume and smaller volume is then the common volume that receives a combination of diffuse electromagnetic radiation and focused ultrasonic radiation. The treatment device 200 also does not necessarily require multiple pluralities of emitters of different modalities. For example, the treatment device 200 can include a first plurality of emitters that are configured to emit radiation of one modality and a single emitter configured to emit radiation of a second modality. The treatment device 200 can also include a single emitter configured to emit radiation of a third modality.

The use of sub-threshold modalities is also not restricted to applications where medicaments or liposomes are activated within the irradiated volume of the head. For example, a treatment device including emitters having different sub-threshold modalities can be used in applications for neural therapies. Neural tissue within the irradiated volume may require a certain power as part of the neural therapy. The emitters may be configured so that the sum of the different modalities is sufficient to provide the required power whilst the power of any one of the modalities is insufficient.

There is also no restriction on the different combinations of modalities required to provide the threshold dose or sufficient power. For example, a treatment device can include emitters of three different modalities of radiation, and any two modalities in combination may be sufficient to provide the required dose. Alternatively, all three modalities may be required to provide the required dose.

Furthermore, the treatment device 200 can include emitters of two or more different modalities, with each individual modality being sufficient to provide the required threshold dose or required power. This will depend on the application of the treatment device.

The treatment device 200 can include emitters of at least the following modalities:
Ultrasonic emitters
Electromagnetic emitters
Magnetic field emitters
Ultrasonic emitters in combination with electromagnetic emitters
Ultrasonic emitters in combination with magnetic field emitters
Electromagnetic emitters in combination with magnetic field emitters
A combination of ultrasonic emitters, electromagnetic emitters, and magnetic field emitters

Any or all of the emitters 204 and/or second emitter(s) and/or third emitter(s) may be adjustable as described herein below.

The orientation and number of first, second, and/or third emitters 204, 214, and 224 can be positioned to intersect their delivered radiation to apply effective acoustic pressure and/or photonic illumination and/or radio frequency radiation and/or magnetic fields to the specified volume 207 inside the head. Calculations can be based on three-dimensional finite element analysis models, CT scans of human cadavers and phantom models, measurements, and MRI thermal imaging, so that emission distribution inside the skull is mapped and predictable with specified tolerances.

In some examples of the treatment device 200, the electromagnetic emitters may include lamps, light emitting diodes; lasers; lenses; transmitters; aerials; and/or coils.

In some examples, the electromagnetic emitters emit electromagnetic radiation at wavelengths selected from the group comprising: narrow; broad; monochrome; visible; infrared; and/or near infrared.

In some examples, the electromagnetic emitters emit radio frequency electromagnetic radiation at wavelengths selected from the group comprising: narrow; broad; short wave; long wave; and/or microwave.

In some examples, the electromagnetic emitters emit electromagnetic radiation at an intensity selected from the group comprising: less than 10 Watt/cm²; less than 1 Watt/cm²; less than 0.1 Watt/cm²; less than 0.01 Watt/cm²; less than 0.001 Watt/cm² less than 0.00001 Watt/cm².

In some examples, the electromagnetic emitters emit radio frequency electromagnetic radiation at an intensity selected from the group comprising: less than 10 Watt/cm²; less than 1 Watt/cm²; less than 0.1 Watt/cm²; less than 0.01 Watt/cm²; less than 0.001 Watt/cm² less than 0.00001 Watt/cm².

The use of an electromagnetic emitter may enable the activation of opto-responsive drugs to regions of the brain, the activation of neurons in the brain, and/or the repair of damage to the brain.

In some examples of the treatment device 200, the magnetic field emitters can include coils; magnets; electromagnets; permanent magnets; inductors; and/or inductor coils.

The use of a magnetic field emitter may enable imaging of the brain, stimulation of neurons in the brain, and/or the activation of magneto-responsive drugs to regions of the brain.

Without being bound by any particular theory, the use of a treatment device to deliver acoustic radiation, electromagnetic radiation, and/or magnetic fields to specified volumes inside the skull may be used for one or more of:
a. Tissue modulation including:
   i. Lesioning tissue
   ii. Opening the blood brain barrier
   iii. Activating specific circuits in the brain
b. The activation of acoustic and/or photonic and/or radio frequency and/or magnetic sensitive agents including:
   i. Contrast agents
   ii. Neuromodulation drugs
   iii. Cytotoxic drugs
c. The treatment of disorders of the brain including:
   i. Degenerative diseases for example: Parkinson's disease, Alzheimer's disease and other dementias
   ii. Malignancy for example: glioblastoma multiforme
   iii. Brain injury for example: stroke; aneurysm; force trauma
   iv. Chronic mental disorder for example: schizophrenia
   v. Mood affective disorders for example: bipolar; depression; PTSD
d. Neural reprogramming including:
   i. Motor skill training, for example: rehabilitation; sports
   ii. Behavioral rectification for example: drug dependency; rehabilitation

### Detectors

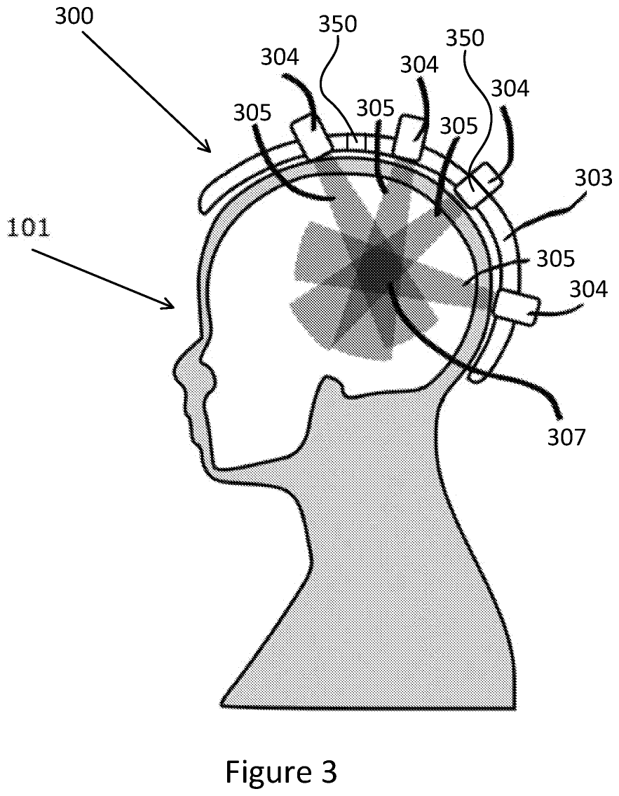

FIG. 3 depicts a further example of a treatment device 300. The treatment device 300 includes a frame 303 and a plurality of emitters 304 supported by frame 303. The plurality of emitters 304 is configured to deliver at least one modality of radiation (depicted by beam 305) to a volume 307 within the head of the wearer of the treatment device. The treatment device 300 can also include one or more second emitter(s) and/or third emitter(s) that are configured to deliver a second and/or third modality of radiation to the volume 307 within the head.

The treatment device 300 further includes a detector 350. The detector 350 is configured to detect at least one modality of signals emanating from within the head of the wearer. The signals may emanate from the volume 307 that is irradiated by the plurality of emitters 305 or may emanate from a different volume. The signal can correspond to the activation and disruption of a medicament or liposome from within the volume 307. The modality of the signal may be independent of the modality (or modalities) of radiation used to activate the medicament. The detector 350 can be an ultrasonic detector and the signal can be an ultrasonic signal.

Detecting a signal associated with the activation of a medicament or liposome can allow for the real-time monitoring of drug release via medicament activation within the target volume 307. This functionality can greatly enhance the utility of the treatment device 300 as it can provide real-time feedback control options and a level of precision and participant safety that would otherwise require a great deal of trial and error and experimentation on each participant.

For example, some configurations of the treatment device 300 can potentially be operated remotely or automatically. In these configurations, the detector 350 can be used as part of a control system to measure the progression of a given treatment and to provide appropriate feedback to the emitters 305 (and/or other emitters if present). For example, the dosage of a drug introduced to the volume 307 via an activated medicament can be inferred or measured by the detector 350 and used to control the characteristics or behaviour of the emitter(s), such as:
Enabling or disabling one or more emitters
Determining which emitters of a given modality should be used (or in other words, enabling one or more emitters on the basis of the modality of their radiation)
Automatically switching off one or more emitters when a sufficient dose has been administered
Adjusting the power, frequency, or other operating characteristic of one or more emitters
Adjusting the position and/or orientation of one or more emitters with respect to the target volume and/or the frame of the treatment device

The data acquired by the detector 350 can also be recorded for later analysis.

A treatment device can also include a plurality of such detectors 350. For example, the number of detectors 350 can be less than 10, 20, 50, 100, 300 detectors. The detectors 350 can be arranged in an array. The array may be sparse or may be dense. The exact geometry of the array will usually depend on the application of the treatment device, such as the location of the target volume 307 within the head and the number of emitters 305 included.

In some examples, the detector 350 can also be integrated with an emitter 305. For example, an ultrasonic detector can be integrated with an ultrasonic emitter. The treatment device 300 can include a combination of emitters 305, detectors integrated with emitters, and dedicated detectors 350.

The plurality of emitters 304 and/or second emitter(s) and/or third emitter(s) may be substantially those disclosed in relation to FIGS. 1 and 2. For example, the treatment device 300 may include:
Ultrasonic emitters
Electromagnetic emitters
Magnetic field emitters
Ultrasonic emitters in combination with electromagnetic emitters
Ultrasonic emitters in combination with magnetic field emitters
Electromagnetic emitters in combination with magnetic field emitters
A combination of ultrasonic emitters, electromagnetic emitters, and magnetic field emitters.

Any or all of the emitters 304 and/or second emitter(s) and/or third emitter(s) may be adjustable as described herein below.

Alternative forms of detectors 350 can also be used with the treatment device 300. Example detectors 350 include ultrasound detectors, piezoelectric detectors, photon detectors, radio frequency radiation detectors, and/or magnetic detectors. Different detectors 350 can also be used in combination within a single treatment device 300.

In some examples, a treatment device can comprise acoustic emitters and/or electromagnetic emitters and/or magnetic emitters that are configured in an adjustable array of emitters, in a frame that is worn on the head, and each said emitter separately delivers radiation to specified volume(s) inside the skull, and that by adjustment of the parameters of the delivered radiation provided by the adjustable array of emitters, the intersection of said illuminated volumes and the combination of radiation at said intersections provides effective focused radiation of single or mixed modality to enable imaging of the brain, lesioning portions of the brain, lesioning malignant tissues in the brain, activating neurons in the brain, repairing damage to the brain, increasing the permeability of the BBB for delivery of materials to the brain, and activating radiation-responsive drugs to specific regions of the brain.

In some examples, a treatment device can comprise a head wearable device incorporating an adjustable array of emitters providing an effective delivery of acoustic radiation and/or electromagnetic radiation and/or magnetic radiation to one or more volumes inside the skull, and adjustment means for changing at least one of the parameters of the delivered radiation provided by the adjustable array of emitters.

In some examples, a treatment can comprise a head wearable device incorporating an adjustable array of emitters providing an effective delivery of acoustic radiation and/or electromagnetic radiation and/or magnetic radiation to one or more volumes inside the skull, an adjustable array of signal detectors providing a detection of acoustic signals and/or electromagnetic signals and/or magnetic signals from one or more volumes inside the skull, and adjustment means for changing at least one of the parameters of the delivered radiation provided by the adjustable array of emitters.

In some examples, a method of delivering acoustic radiation and/or electromagnetic radiation and/or magnetic radiation to one or more specified volumes inside the skull can comprise: providing a head wearable device incorporating an array of emitters configured to emit acoustic radiation and/or electromagnetic radiation and/or magnetic radiation; adjusting the array of emitters to a prescribed geometry and/or direction and/or intensity to change at least one of the parameters of the radiation and causing effective radiation to be focused on specified volumes inside the skull.

In some examples, a method of manufacturing a treatment device can comprise:
a. Determining a Three-dimensional Topology of a Head;
b. determining a modality, number, and orientation of emitters required to deliver effective radiation to specified volume inside the skull; and
c. manufacturing a head wearable frame that fits the topology of the head, positions emitters to deliver effective radiation to specified volumes inside the skull, and optionally positions signal detectors to effectively detect signals emanating from inside the skull.

In some uses of example treatment devices, control of an adjustment of delivered radiation can be effected on the basis of one or more parameters selected from the group comprising: a modality of emitters; a number of emitters; a placement of the emitters; a direction of the emitters; a modality of the radiation; a direction of the radiation; an intensity of the radiation; a power of the radiation; an amplitude of the radiation; a phase of the radiation; a timing of the radiation; and/or a frequency of the radiation.

### Adjustable Treatment Devices

Some treatment devices may have emitters that are fixed in a static location with respect to the frame of the treatment device. Alternatively or additionally, the treatment device can include adjustable emitters. This can allow the emitters of the treatment device to target different regions and volumes within the head of the wearer, thereby increasing the versatility of the treatment device.

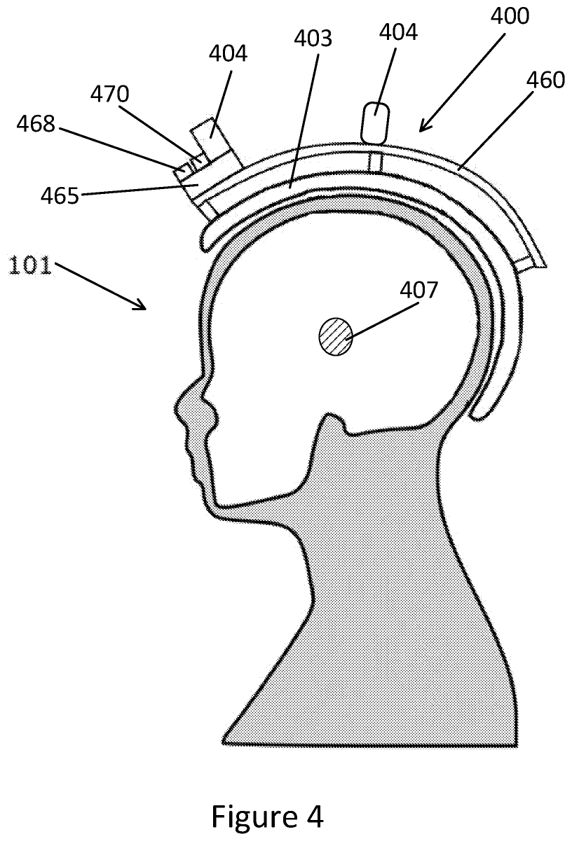

FIG. 4 depicts an example of a treatment device 400. The treatment device 400 includes a frame 403 that is configured to be worn on a head. The frame 403 supports a plurality of emitters 404 that are configured to deliver at least one modality of radiation to a volume 407 within the head of the wearer 101. The treatment device 400 is configured so that an orientation and/or position of at least one emitter 404 with respect to the frame 403 can be adjusted. The plurality of emitters 404 are configured to deliver radiation to a common volume 407. The plurality of emitters 404 can be configured to activate a liposome within the volume 407.

In this particular example, the frame 403 includes one or more rails or guides 460. One or more emitters 404 can be secured to the rail or guide 460 at different positions on the frame 403, thereby allowing for the adjustment of the position of the one or more emitters 404 with respect to the wearer's head 101.

For example, the one or more emitters 404 can be releasably or permanently secured on a carriage 465 that is able to slide along the rail or guide 460. The carriage 465 can be fixed at a position on the rail or guide 460 to set the position of the associated emitter 404. Some rails 460 may allow for the carriage 465 to be fixed at any position along the rail 460, whilst other rails 460 may allow for the carriage 465 to be fixed at one or more discrete positions. The carriage 465 may include a servo motor 468 to drive the carriage 465 along the rail 460 and set its position. Stepper motors or the like can also be used instead of servo motor 468. A piezoelectric element 470 can additionally or alternatively be used to enable fine adjustment of the position of the carriage 465 on the rail 460. The carriage 465 may alternatively or additionally be manually adjustable about the rail 460.

The frame 403 may include multiple rails 460 and carriages 465. The multiple rails 460 may be approximately orthogonal to one another.

Some treatment devices 400 may include a combination of permanently fixed emitters 404 and adjustable emitters 404. The emitters 404 may be pivotable or rotatable about one or more axes with respect to the carriage 465. This means that the angle and direction of the emitter 404 can be adjusted with respect to the head of the wearer 101 and the volume 407. The emitter 404 may be manually pivotable about carriage 465 (for example, using a gimbal-like mechanism) and/or may be driven by piezoelectric actuators or the like. This can allow for very precise adjustment to the emission direction of the emitter.

The treatment device 400 may include one or more detectors as described herein, particularly with reference to FIG. 3. Furthermore, the treatment device 400 may include any of the varieties of the emitters described herein, either alone or in combination. For example, the treatment device may include:
Ultrasonic emitters
Electromagnetic emitters
Magnetic field emitters
Ultrasonic emitters in combination with electromagnetic emitters
Ultrasonic emitters in combination with magnetic field emitters
Electromagnetic emitters in combination with magnetic field emitters
A combination of ultrasonic emitters, electromagnetic emitters, and magnetic field emitters

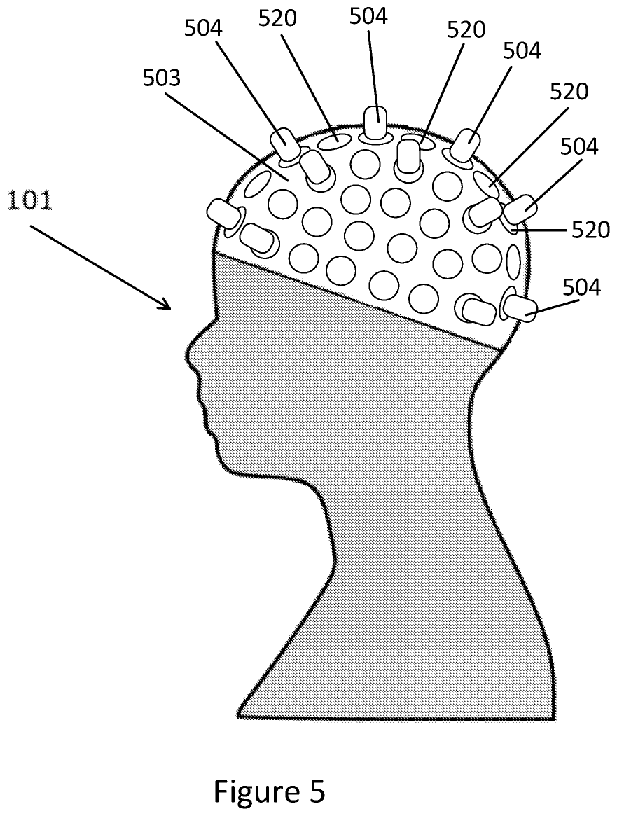

FIG. 5 depicts a further example of an adjustable treatment device 500. This treatment device 500 includes a frame 503 that has a plurality of apertures 520 that are spaced about the frame 503 and are configured to receive one or more emitters 504. The number of emitters 504 incorporated into the frame 503 and their initial position with respect to the frame 503 are determined during the assembly or construction of the treatment device 500. The emitters 504 are preferably removable from the apertures 520 of the frame 503 so that the number and/or position of the emitters 504 can be altered after the treatment device 500 has first been assembled. Each emitter 504 is also preferably pivotable about one or more axes within its aperture so that the direction of emission can be adjusted.

The treatment device 500 may include one or more detectors as described herein, particularly with reference to FIG. 3. Furthermore, the treatment device 500 may include any of the varieties of the emitters described herein, either alone or in combination. For example, the treatment device may include:
Ultrasonic emitters
Electromagnetic emitters
Magnetic field emitters
Ultrasonic emitters in combination with electromagnetic emitters
Ultrasonic emitters in combination with magnetic field emitters
Electromagnetic emitters in combination with magnetic field emitters
A combination of ultrasonic emitters, electromagnetic emitters, and magnetic field emitters

### Manufacture of Treatment Devices

The required number, modality, and position/orientation of the emitters of a given treatment device can vary depending on the application of the treatment device. For example, two different participants may require different modalities of emissions at two different volumes within their respective heads. Similarly, differences in head or skull shape between people can also affect the required configuration of the frame and/or the emitters of the treatment device. For example, the diffraction and reflection of ultrasonic radiation within a head can be affected by the shape of the skull and the distribution of different materials (e.g. bone, fat, and other tissues) within the head. It can therefore be advantageous to create customised treatment devices for use with a given head and for a specific application.

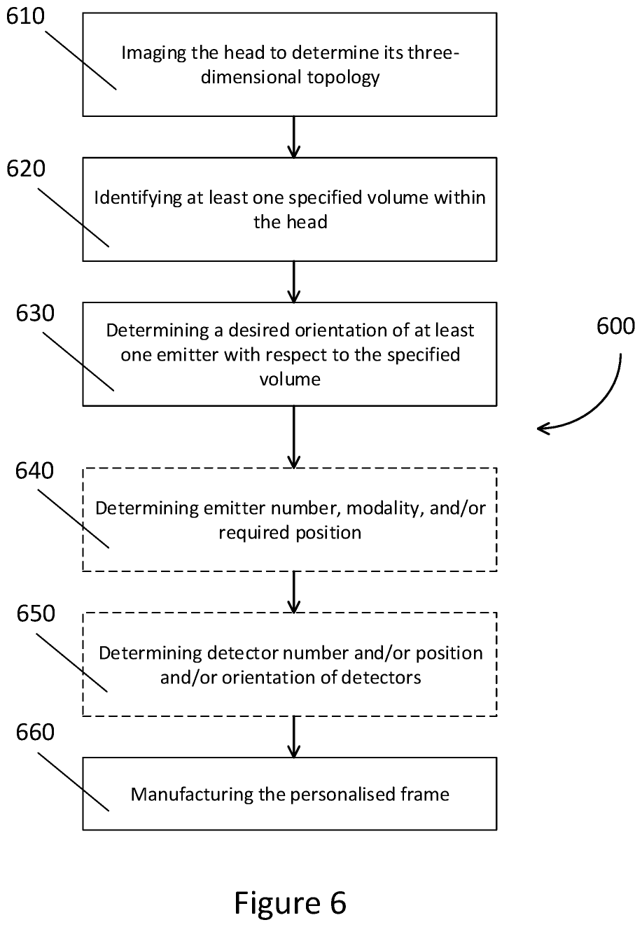

FIG. 6 depicts an example of a method 600 of manufacturing a customised treatment device. The head of the wearer is first imaged at 610 to determine a three-dimensional topology of the head. A number of different imaging techniques can be used including measuring; imaging; scanning; computerized tomography (CT) scanning; x-ray; magnetic resonance imaging (MRI); optical scanning; magnetoencephalography (MEG); and/or positron emission tomography (PET) scanning, either alone or in combination and with or without contrast agents.

At least one specified volume that is to be irradiated by a plurality of emitters is then identified from the three-dimensional topology of the head at 620. Multiple volumes may be identified depending on the required application of the treatment device. A desired orientation of at least one emitter with respect to the at least one specified volume is then determined on the basis of the specified volume at 630. The required number of emitters, modality of emitters, and/or their required position with respect to the at least one specified volume can also be determined at 640 if required. Finite element modelling can be used to determine the arrangement and configuration of the emitters. The emitters may be arranged so that they will deliver radiation to a common volume that is identified within the head using the aforementioned imaging of the head. The emitters can be further arranged so that the common volume will receive a threshold dose (e.g. to activate a medicament or liposome) whilst off-axis volumes will receive a sub-threshold dose. A number of required or desired detectors and their position and/or orientation may also be determined at 650 if required.

Once the desired orientation of the at least one emitter with respect to the specified volume has been determined at 630, a customised frame that is configured to be worn on the head is then manufactured at 660. The customised frame is configured to support the at least one emitter such that when the customised frame is worn on the head, the at least one emitter is positioned with the desired orientation with respect to the at least one specified volume within the head.

In some examples, the customised frame may be manufactured using 3D printing or other additive manufacturing techniques. This can allow for comparatively rapid manufacture of precision frames that are highly customised and yet are comparatively low-cost to produce. Other forms of manufacturing are also possible, such as precision subtractive manufacturing. The customised frame may be a skeletonized exo-skullcap that is engineered to cradle orient the required emitter-detectors.

Scaling of the three-dimensional model and re-printing of the frame enables variations in head size to be accommodated and for re-targeting to alternative specific volumes inside the skull. For example, a given participant may require multiple treatments at different treatment locations (due to e.g. multiple lesions located at the treatment locations) or at different times (e.g. receiving a first modality of radiation and subsequently receiving a second modality of radiation at a later time.) A series of customised frames can be manufactured for that particular participant such that each individual frame is configured for treatment at a particular location and/or time.

For example, a first customised frame can be configured to support one or more emitters at an orientation that targets a first treatment location. A second customised frame can be configured to support one or more emitters at an orientation that targets a second treatment location. The participant can then use the first customised frame to receive treatment at the first location and can subsequently use the second customised frame to receive treatment at the second location. Both the first and second customised frames are customised for the participant (e.g. can be configured to match the exterior contours of the participant's head) but each individual frame is configured for a particular treatment of the participant.

The position and orientation of the emitter with respect to the customised frame and the shape of the customised frame can not only account for the overall shape of the wearer's head, but can also account for the composition of the wearer's head.

For example, the propagation of ultrasonic radiation through the wearer's head is influenced by diffraction and reflection at the interfaces of different kinds of tissues, such as muscle and bone. These interfaces can be numerous and can have complex shapes that vary from head to head. Accurately placing an ultrasonic emitter at the right position and with the right orientation with respect to the target volume can therefore require knowledge of the external contours of the wearer's head (i.e. their scalp) and also the internal composition of their head.

Finite element modelling may be used to model how ultrasonic radiation will propagate through the wearer's head. The position and orientation of an ultrasonic emitter can then be determined such that when the ultrasonic emitter is at the desired position and orientation, the ultrasonic radiation will propagate such that the at least one identified volume is irradiated with its desired characteristics.

The manufactured treatment device may include one or more detectors as described herein, particularly with reference to FIG. 3. Furthermore, the manufactured treatment device may include any of the varieties of the emitters described herein, either alone or in combination. Any or all of these emitters may be adjustable as described herein. The manufactured treatment device may include:
Ultrasonic emitters
Electromagnetic emitters
Magnetic field emitters
Ultrasonic emitters in combination with electromagnetic emitters
Ultrasonic emitters in combination with magnetic field emitters
Electromagnetic emitters in combination with magnetic field emitters
A combination of ultrasonic emitters, electromagnetic emitters, and magnetic field emitters

### Supporting Experiments

To confirm the headset irradiation of specified volumes inside the skull, the applicant performed experiments including:

Acoustic mapping of the ultrasound beam profiles of the emitter-detectors individually and in the sparse array; with an anatomically realistic three-dimensional model head; and with cadaver heads. High resolution CT head scans of cadaver heads were performed to establish anatomical compensation and bespoke positioning of the treatment device for ultrasound emission and detection measurements. In some cases, this may be done just prior to tissue fixation (required for long term cadaver storage), to permit comparison of the emission received with and without acoustic-sensitive liposomes injected into the brain vasculature. Post-stimulation, CT scans and anatomical dissection were performed. The brain was removed intact (craniotomy), and a tissue-specific ultrasonic hydrophone probe inserted into each hemisphere within the striatum volume. The brain was returned to the cranial vault, the cranium restored, and the headset reinstalled. Ultrasound emissions measured by the hydrophone in the dissected tissue were used to validate and calibrate model predictions.

Skull studies using a HIFU 256 channel Verasonics system with three-dimensional scanning tank system for precise determination of the acoustic pressure magnitudes and profiles, and for the development and validation of phase steering methods.

Using liposomes containing dopamine agonists in neurotoxin-lesioned sheep (parkinsonian model). Sheep preparation comprises: i) CT head scanning to determine emitter coordinates; ii) lesion surgery of the substantia nigra (injection of toxin bilaterally but asymmetrical volumes to promote asymmetric turning and improve welfare) and recovery; iii) systemic DA challenge (apomorphine, dihydrexidine and ropinirole) to verify the lesion and sensitivity; iv) emitter placement on animals; v) intravenous cannulation; vi) behavioural assessment. At the end of the experiments, brains were harvested to determine catecholamine levels (dopamine, homovanillic acid and dihydoxyphenylacetic acid) by high performance liquid chromatography, and immunohistochemistry to determine the loss of cells in each substantia nigra. Following bilateral lesioning, all sheep appeared slow in movement, but otherwise able to mobilize and graze. Sheep also showed spontaneous turning if the dopamine depletion was asymmetrical.

Using liposomes containing dopamine agonists in lesioned sheep, we can selectively enhance the function of one striatum at a time, to cause asymmetric turning. This allows demonstration of precise control of the system over separate (but similar functioning) brain areas. This may provide an internal control (lesioned vs non-lesioned) and comparison with non-operated animals administered liposomes containing dopamine antagonists to effectively switch off the target area. Sheep preparation can comprise: i) CT scanning the head to determine transducer coordinates; ii) in operated animals lesion surgery of the substantia nigra (injection of toxin bilaterally but asymmetrical volumes to promote asymmetric turning and improve welfare) and recovery; iii) emitter placement on animals; iv) intravenous cannulation; v) behavioural assessment. We will underpin these experiments with prior rat testing to verify asymmetric turning on projecting ultrasound emissions on one side of the brain.

Behavioural assessment (incorporating ultrasound detection recording) in the paddock comprised harnessing the sheep to carry an emitter controller-recorder electronics and GPS telemetry. The experimental session lasted 3-4 hours, involving ultrasound application in the absence and presence of circulating neuromodulator-loaded liposome. Behaviour was measured against control pen mates using a satellite videography to record all gross movements. The presence of asymmetrical turning showed that the head-mounted emitters applied sufficient acoustic pressure to a specific volume inside the skull to achieve threshold activation of acoustic-sensitive liposomes.

High resolution MRI scanning was used to validate headset projection of ultrasound signal, by applying ultrasound and injected microbubbles sufficient to open the BBB in the presence of the MRI contrast agent gadolinium. After the ultrasound had been applied, the headset was removed, and the head scanned to determine the extent and location of the extravasation of contrast medium.

Real-time drug release was monitored by detecting liposome-induced ultrasound emissions emanating from the focal region.

A mixed modality to trigger drug release from acoustic-sensitized liposomes comprising ultrasound combined with near infrared (NIR), to offer additional functionality to enhance non-invasive drug release at sub-threshold acoustic pressures. A mixed modality drug release paradigm that combined monochromatic infrared radiation with ultrasound pressure showed incorporation of near-infrared sensitizing features in our ultrasound sensitive liposomes (e.g. the dye IR780 incorporated into the liposome bilayer) may cause disruption and drug release upon near-infrared illumination and ultrasound independently and together.

### Ultrasonic Emitters

Treatment devices can use ultrasonic emitters that do not require surgical implantation. These can deliver ultrasonic radiation through the scalp and scalp/skull interface of the wearer to a volume (e.g. a part of the brain) within the wearer's head. The applicant has found that the scalp-skull-brain pathway of a head can have poor acoustic transfer properties for the delivery of ultrasonic radiation to the brain for neural treatment. Acoustic losses can particularly occur at the interface of the scalp and the skull of the wearer. Conventional ultrasonic emitters that are not designed to minimise these losses can be unsuited or less desirable for use in wearable treatment devices.

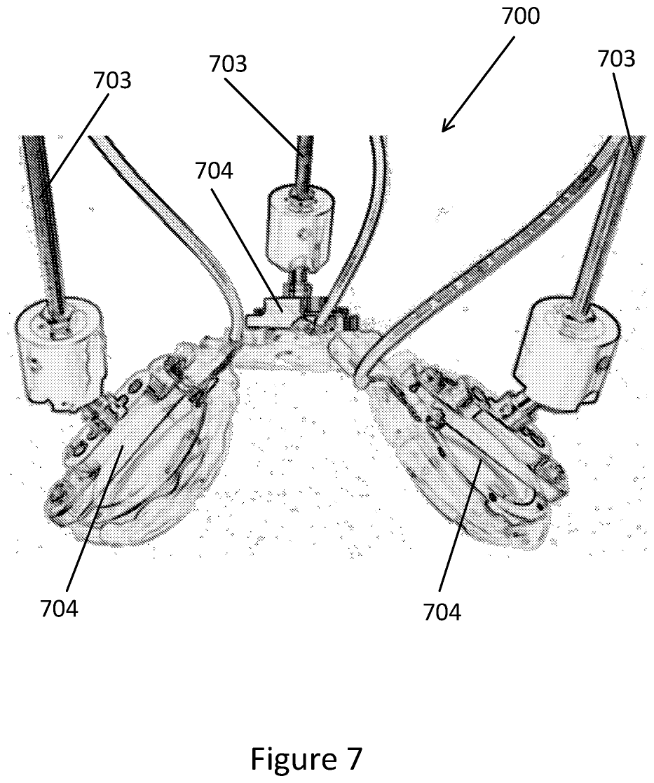

FIG. 7 depicts an example of a treatment device 700 comprising an adjustable frame 703 (only partially shown) supporting three ultrasonic emitters 704. The ultrasonic emitters 704 are configured to deliver ultrasonic radiation to a volume within the head of the wearer through the wearer's scalp/skull interface. Losses at the scalp/skull interface can be minimised if the ultrasonic emitters are configured to establish a reverberation with the scalp/skull interface to match the impedance of the interface. The operating frequency of the emitters can be at least partially chosen to improve the transmission of ultrasonic radiation through the skull of the wearer. Other design factors (such as the type of treatment required by the wearer) can also at least partially dictate the operating frequency of the ultrasonic emitters.

The ultrasonic emitters 704 can be small in size and generally dimensioned for use in convenient head-wearable treatment devices. The ultrasonic emitters 704 depicted in FIG. 7 have a diameter of 35 mm, a thickness of 10 mm, and weigh 45 grams. The electronic circuitry used to control the treatment device 700 (not depicted) also can have form factor that is portable in size (for example, a footprint of approximately 8 centimetres by 5 centimetres or less.) The control of the treatment device 700 can be prescribed entirely by the hardware implantation of the control circuitry so that a microprocessor or computer is not required. Supervisory functions can be built into the control circuitry so that the treatment device 700 is configured to be fail-safe.

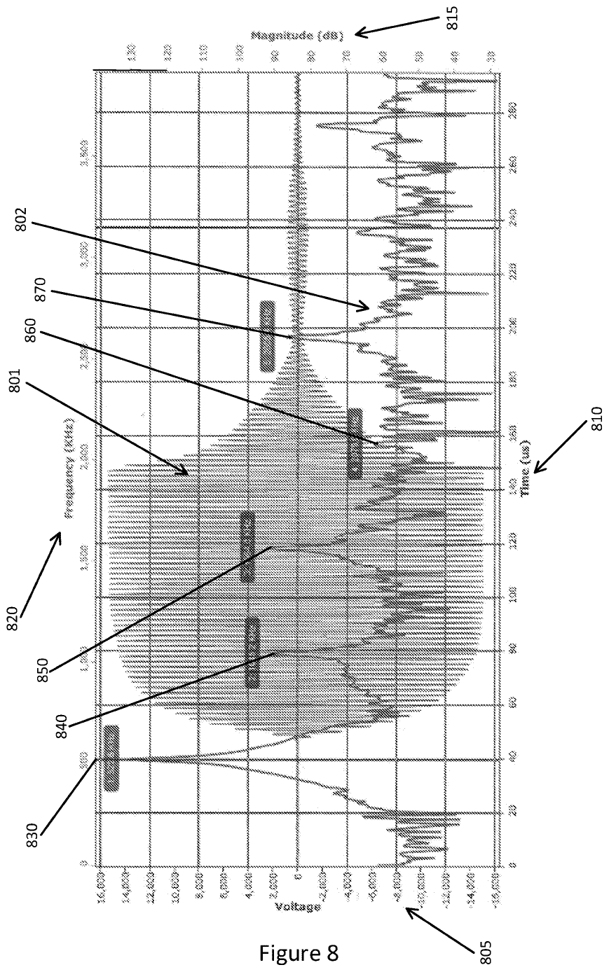

The ultrasonic emitters were characterised by measuring the emitted ultrasonic radiation using a three-dimensional scanning tank system and hydrophone. FIG. 8 shows the signal 801 measured by the hydrophone in the time domain. The signal of the hydrophone is expressed in units of volts on y-axis 805 against time (in units of microseconds) on axis 810.

FIG. 8 also shows the hydrophone's signal 802 in the frequency domain in units of dB on y-axis 815 against frequency (in units of KHz) on x-axis 820. The output of the ultrasonic emitter has a fundamental frequency of approximately 520 KHz as indicated by peak 830. Second-order, third-order, and fifth-order harmonics are indicated by 840, 850, and 870, respectively. These peaks have an amplitude of approximately −45 dB relative to the amplitude of the fundamental frequency. The magnitude of the fourth-order harmonic 860 does not appear to be substantially above the noise floor of the signal in the frequency domain.

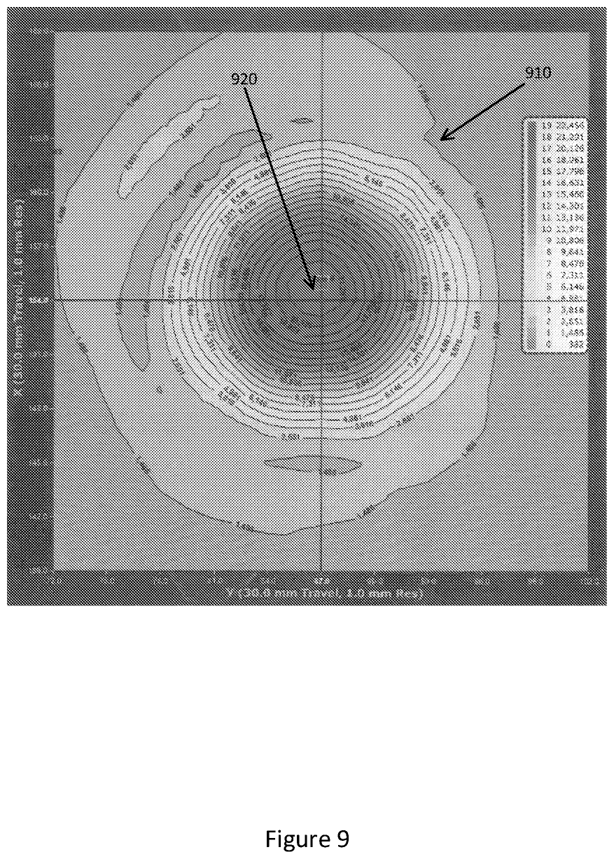

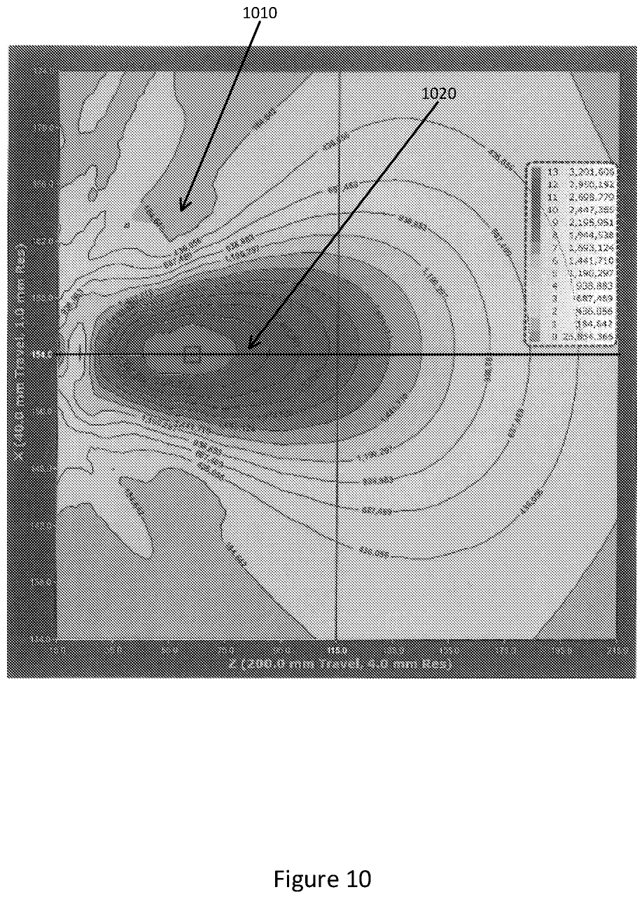

FIGS. 9 and 10 depict contour maps showing the sound pressure level at different positions with respect to one of the ultrasonic emitters. FIG. 9 is a lateral plot showing the measured sound pressure level in the x-y plane at a fixed depth (i.e. z coordinate.) The measured sound pressure (generally indicated by 910) is substantially symmetric about the axis of emission 920 (which is ‘into’ or ‘out of’ the page from the perspective of FIG. 9).

FIG. 10 is an axial plot showing the sound pressure level in the x-z plate at a fixed y-coordinate. The measured sound pressure 1010 is substantially symmetric about the axis of emission 1020.

The ultrasonic emitter's capability to transmit ultrasonic radiation through a scalp/skull interface was characterised using a True Phantom Solutions phantom head. The phantom head has an anatomy that mimics an average human head (including a scalp and skull) and is also made of materials that mimic the acoustic properties of an average human head.

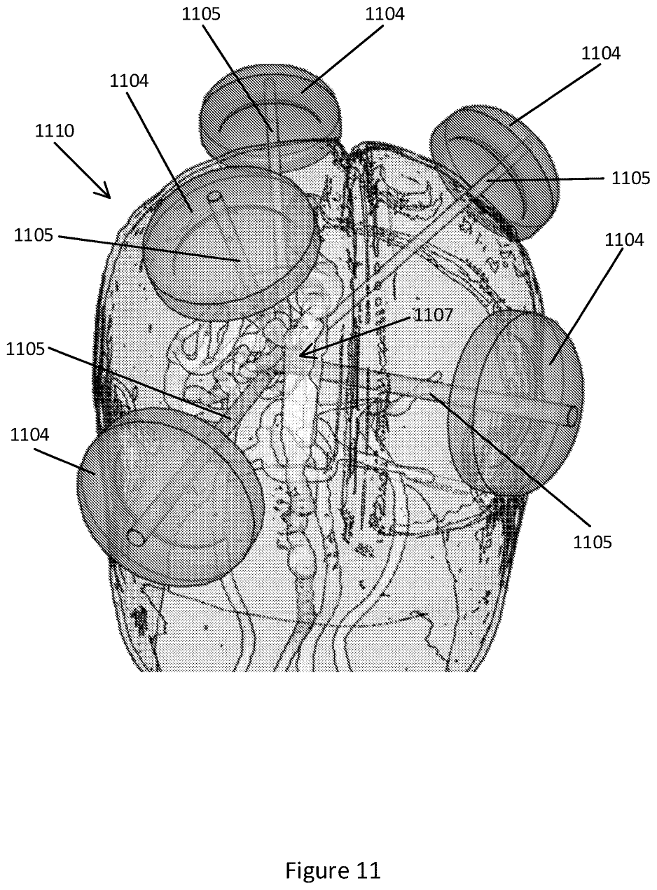

FIG. 11 depicts a desired orientation of a number of emitters with respect to the phantom head 1110. The phantom head 1110 was imaged to determine its three-dimensional topology. A specified volume (indicated by 1107) to be treated was identified from the three-dimensional topology of the phantom head 1110. Modelling was used to determine the desired orientation of five ultrasonic emitters 1104 such that the ultrasonic emitters 1104 would irradiate volume 1107. The axes of emission 1105 of each ultrasonic emitter 1104 are generally orthogonal to one another in this desired orientation and intersect at common volume 1107 that is to receive treatment. The ultrasonic radiation must pass through the scalp and scalp/skull interface of the phantom head 1110 to reach the volume 1107.

Ultrasonic emitters were intentionally chosen for this experiment in order to characterise their ability to transmit ultrasonic radiation through a scalp/skull interface. In other examples, the modality of the emitters could have been determined at least partially based on the imaged topology of phantom head 1110 and the identified volume 1107. Similarly, whilst five ultrasonic emitters 1104 were chosen for this experiment, the number of emitters could have also been determined depending at least partially on the imaged topology of the phantom head 1110, the nature of the volume 1107 (including, for example, the size, shape, location, and/or contents of the volume 1107), and the nature of the treatment required.

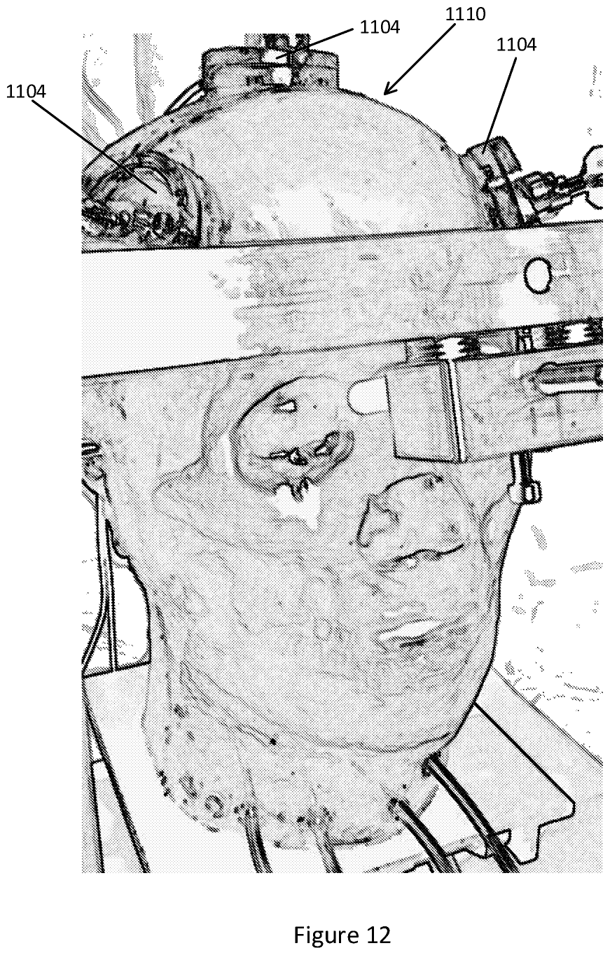

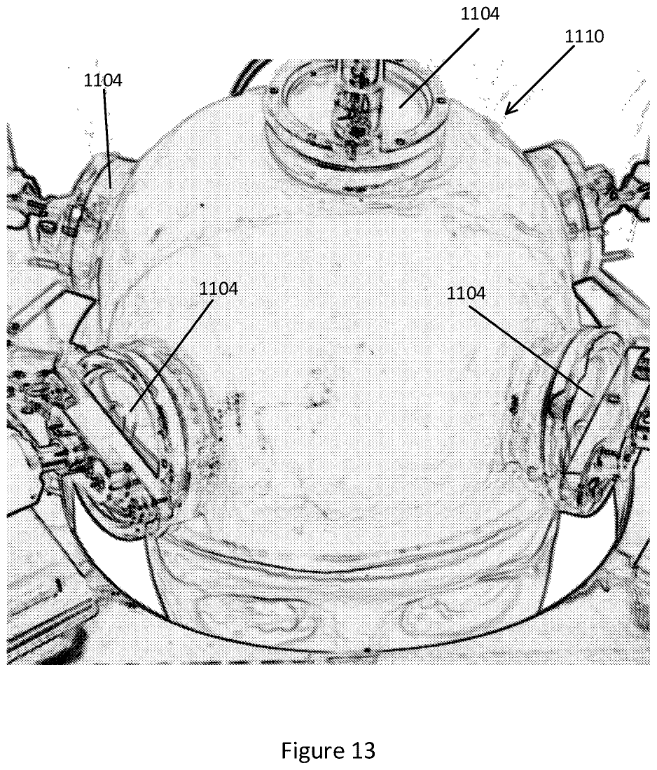

A customised frame configured to support the emitters 1104 in their desired orientation about the phantom head 1110 was then manufactured using the imaged topology of the phantom head 1110. FIGS. 12 & 13 show the treatment device as worn atop the phantom head 1110. The ultrasonic emitters 1104 are in contact with the exterior scalp of the phantom head 1110 and are positioned with the desired orientation with respect to the volume 1107 within the head (shown in FIG. 11). In this case, the customised frame surrounds the phantom head 1110. The customised frame supporting emitters 1104 could alternatively be 3D printed, constructed using other additive manufacturing techniques, or any of the techniques described herein using the imaged topology. A series of customised frames can also be constructed for the phantom head 1110 if, for example, other volumes within the head required treatment, if the same volume 1107 required treatment from different emitter orientations (e.g. if the volume 1107 was particularly large), or if further treatment was required at a later stage. The treatment device could also have included detectors, different modalities of emitters in isolation or in combination, and/or one or more adjustable emitters, as described herein.

The ultrasonic emitters 1104 were used to irradiate the volume 1107 and the acoustic pressure at the volume 1007 was measured to determine the transmission of ultrasonic radiation through the scalp/skull interface. Losses at the scalp/skull interface were reduced due to impedance matching through reverberation at the scalp/skull interface. Data from the experiment suggests that refraction by the skull can affect the depth of focus of each ultrasonic emitter. This can be accounted for to achieve the desired depth of focus. Acoustic signal losses caused by wave scattering via skull porosity was notably less than wave scattering signal losses in sheep skulls.

### Advantages

Amongst the advantages already described above, disclosed herein are treatment devices that can deliver ultrasound pressure and/or photonic illumination and/or radio frequency radiation and/or magnetic fields in single or mixed modalities of emissions to specific volumes inside the skull. Arrays of emitters can permit sub-threshold radiation to be delivered from separate emitters to volumes inside the skull wherein only where intersecting radiation from multiple emitters are at or above threshold, thereby minimizing non-specific effects. The example treatment devices do not require surgical installation, can deliver continuous or semi-continuous or on-demand acoustic pressure and/or photonic illumination and/or radio frequency radiation and/or magnetic fields to specified volumes inside the skull, can be conveniently reconfigured to alternative volumes inside the skull, can be conveniently serviced, and are comparatively inexpensive to manufacture to enable their widespread availability.

Some examples of treatment devices may be used to provide remote medical treatment in communities that are far from resources, or in times when there are contagions (such as Covid-19) that limit personal travel. Some examples of treatment devices can also potentially be used in communities where surgical interventions of the head are culturally difficult. The use of a detector to enable automatic control of the treatment device can also allow for treatment in non-conventional circumstances. For example, in some instances, the frame of the treatment device can be an otherwise innocuous article of clothing or an accessory (e.g. a pair of glasses) that can deliver treatment at convenient times, such as on the bus or in the home. In comparison, conventional treatments may need to be performed in specialised facilities with the direct supervision of medical specialists.

The use of a treatment device may overcome several of the disadvantages present in conventional precision surgical treatment of the brain for lesioning portions of the brain, repairing damage to the brain, removing malignant tissues, or inserting electrodes for stimulation of the brain. These conventional surgeries can be highly invasive and carry many risks associated with craniotomy, which is the surgical removal of part of the skull, to gain access to the brain. Surgical risks include for example blood loss, tissue damage, infection, and adverse reactions to anaesthetic drugs.

While the present invention has been illustrated by the description of the examples thereof, and while the examples have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

1. A treatment device configured to be worn on a head, the treatment device comprising:
a frame configured to be worn on a head, and
a plurality of emitters supported by the frame, the plurality of emitters configured to deliver ultrasonic radiation to a volume within the head;
wherein the plurality of emitters is configured to activate a medicament cause release of a medicament, activate microbubbles, or for neuromodulation within the volume irradiated by the emitters.

2. The treatment device of claim 1, wherein the treatment device further comprises a plurality of second emitters configured to deliver a modality of magnetic radiation to the volume within the head.

3. The treatment device of claim 1, wherein the treatment device further comprises a plurality of third emitters configured to deliver a modality of electromagnetic radiation to the volume within the head.

4. The treatment device of claim 1, wherein the treatment device further comprises at least one detector selected from an ultrasonic detector, a magnetic detector or an electromagnetic detector configured to detect at least one modality of signals emanating from the head.

5. The treatment device of claim 4, wherein said at least one detector is integrated with an emitter.

6. The treatment device of claim 4, wherein said detector is configured to monitor drug release within the volume within the head.

7. The treatment device of claim 6, wherein the device further comprises a control system configured to measure the progression of a given treatment.

8. The treatment device of claim 7, wherein the control system provides feedback to at least one emitter.

9. The treatment device of claim 8, wherein the feedback is used to enable or disable one or more emitters.

10. The treatment device of claim 1, wherein the one or more emitters are enabled or disabled on the basis of their modality of radiation.

11. The treatment device of claim 8, wherein the feedback is used to automatically turn off one or more emitters when a sufficient dose of radiation has been administered.

12. The treatment device of claim 8, wherein the feedback is used to adjust the position and/or orientation of one or more emitters with respect to the volume and/or the frame of the treatment device.

13. The treatment device of claim 1, wherein the orientation and/or position of at least one emitter can be adjusted with respect to the frame.

14. The treatment device of claim 1, wherein the treatment device is a portable device.

15. The treatment device of claim 14, wherein the treatment device is untethered.

16-66. (canceled)
